(19) United States
(12) Patent Application Publication
(10) Pub. No.: US 2025/0152556 A1
(43) Pub. Date: May 15, 2025

(54) ETRASIMOD FOR USE IN TREATING S1P1 RECEPTOR-ASSOCIATED DISORDERS IN COMBINATION WITH HORMONE TREATMENT

(71) Applicant: Arena Pharmaceuticals, Inc., New York, NY (US)

(72) Inventors: John Stewart Grundy, Carlsbad, CA (US); Caroline A. Lee, Carlsbad, CA (US)

(73) Assignee: Arena Pharmaceuticals, Inc., New York, NY (US)

(21) Appl. No.: 18/722,707

(22) PCT Filed: Jan. 9, 2023
(86) PCT No.: PCT/IB2023/050173

§ 371 Date: Jun. 21, 2024

Related U.S. Application Data
(60) Provisional application No. 63/299,251, filed on Jan. 13, 2022.

(51) Int. Cl. A61K 31/403; A61K 31/565; A61P 15/18

(52) U.S. Cl. CPC A61K 31/403; A61K 31/565; A61P 15/18

(57) ABSTRACT

Provided is (R)-2-(7-(4-cyclopentyl-3-(trifluoromethyl)benzyloxy)-1,2,3,4-tetrahydrocyclopenta[b]indol-3-yl)acetic acid (Etrasimod, Compound 1), or a pharmaceutically acceptable salt, hydrate, or solvate thereof for use in a method of treatment of an S1 P1 receptor-associated disorder in a subject, said method comprising administering said compound to said subject, wherein the a subject is also being administered a hormone treatment.

Etrasimod, or a pharmaceutically acceptable salt, hydrate, or solvate thereof, such as L-arginine etrasimod, is an oral, selective sphingosine 1-phosphate receptor 1, 4, 5 modulator, and is in development for ulcerative colitis, atopic dermatitis, alopecia areatea, eosinophilic esophagitis, and Crohn's disease. To date, etrasimod, or a pharmaceutically acceptable salt, hydrate, or solvate thereof, has been found to be safe and well-tolerated in approximately 281 adult subjects treated at various doses. Its safety and tolerability have been evaluated in Phase 1 studies with healthy adult subjects at single doses up to 5 mg and repeated doses up to 4 mg once daily (QD). In a Phase 2 dose-ranging study in ulcerative colitis patients, treatment with 2 mg QD for 12 weeks led to clinically meaningful and statistically significant endoscopic and symptomatic improvements versus placebo. Sustained beneficial effects of were observed for up to 46 weeks in the subsequent open-label extension study.

Estrogen is one of two main sex hormones that women have. The other one is progesterone. Estrogen is responsible for female physical features and reproduction. Men have estrogen, too, but in smaller amounts. There are three major endogenous estrogens that have estrogenic hormonal activity: estrone (E1), estradiol (E2), and estriol (E3). Estradiol, an estrane, is the most potent and prevalent. Another estrogen called estetrol (E4) is produced only during pregnancy. In addition to their role as natural hormones, estrogens are used as medications, for instance in menopausal hormone therapy, hormonal birth control and feminizing hormone therapy for transgender women and nonbinary people.

LO LOESTRIN®, a combination of norethindrone acetate and ethinyl estradiol for use by women to prevent pregnancy, had about $356 million of sales in 2020. YAZ®, a combination of drospirenone and ethinyl estradiol for use to prevent pregnancy, treat symptoms of premenstrual dysphoric disorder, and to treat moderate acne, had about $810 million in sales.

There exists a need for safely treating individuals who are in need of treatment with etrasimod 1, or a pharmaceutically acceptable salt, hydrate, or solvate thereof, including individuals being administered a hormone treatment, such as an estrogen and/or a progesterone. The present disclosure satisfies this need and provides related advantages as well.

### SUMMARY

Provided is a method of treatment of an S1P1 receptor-associated disorder in a subject, the method comprising administering (R)-2-(7-(4-cyclopentyl-3-(trifluoromethyl)benzyloxy)-1,2,3,4-tetrahydrocyclopenta[b]indol-3-yl)acetic acid (Compound 1 or etrasimod or a pharmaceutically acceptable salt, hydrate, or solvate thereof, such as Compound 2, to said subject, wherein the subject is also being administered a hormone treatment.

These and other aspects of the invention disclosed herein will be set forth in greater detail as the patent disclosure proceeds.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” means (R)-2-(7-(4-cyclopentyl-3-(trifluoromethyl)benzyloxy)-1,2,3,4-tetrahydrocyclopenta[b]indol-3-yl)acetic acid, including crystalline forms thereof.

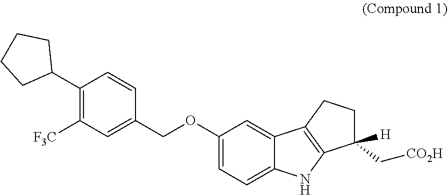 [SMILES: O=C(O)C[C@H]1CCc2c1[nH]c1ccc(OCc3ccc(C4CCCC4)c(C(F)(F)F)c3)cc21]

See PCT patent application, Serial No. PCT/US2009/004265 hereby incorporated by reference in its entirety. As a non-limiting example, Compound 1 may be present as an anhydrous, non-solvated crystalline form as described in WO 2010/011316 (incorporated by reference herein in its entirety). Compound 1 is referred to in literature as etrasimod or APD334.

Methods of use of Compound 1, or a pharmaceutically acceptable salt, hydrate, or solvate thereof, are disclosed in US Patent Application Publication No. 2018-0263958, PCT Publication No. WO2021/102357, PCT Publication No. WO2021/067506, and PCT Patent Application No. PCT/US2021/012367, which are hereby incorporated by reference in their entirety.

COMPOUND 2: As used herein, “Compound 2” means the 1:1 salt of (R)-2-(7-(4-cyclopentyl-3-(trifluoromethyl)benzyloxy)-1,2,3,4-tetrahydrocyclopenta[b]indol-3-yl)acetic acid with L-arginine, including crystalline forms thereof. Compound 2 may be present as an anhydrous, non-solvated crystalline form as described in WO 2010/011316 and WO 2011/094008 (each of which is incorporated by reference herein in its entirety).

ADMINISTERING: As used herein, “administering” means to provide a compound or other therapy, remedy, or treatment such that an individual internalizes a compound.

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to an individual. In this case the health care practitioner may or may not provide a prescription for the compound, dosage regimen, or treatment. Further, the health care practitioner may or may not provide the recommended compound or treatment. For example, the health care practitioner can advise the individual where to obtain the compound without providing the compound. In some embodiments, a health care practitioner can provide a prescription for the compound, dosage regimen, or treatment to the individual. For example, a health care practitioner can give a written or oral prescription to an individual. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the individual. As used herein, giving a sample of a compound constitutes an implicit prescription for the compound. Different health care systems around the world use different methods for prescribing and/or administering compounds or treatments and these methods are encompassed by the disclosure.

A prescription can include, for example, an individual's name and/or identifying information such as date of birth. In addition, for example, a prescription can include: the medication name, medication strength, dose, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a condition described herein. In addition, a healthcare practitioner can include anyone who can recommend, prescribe, administer, or prevent an individual from receiving a compound or drug including, for example, an insurance provider.

PREVENT, PREVENTING, OR PREVENTION: As used herein, the term “prevent,” “preventing”, or “prevention,” such as prevention of a particular disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to an individual who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein, the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting, even if the severity of each is less than when experienced alone.

INTOLERANCE: As used herein, “intolerance” means significant toxicities and/or tolerability issues that led to a reduction in dose or discontinuation of the medication. “Intolerance” can be replaced herein with the term “unable to tolerate.”

ADVERSE EVENT: As used herein, an “adverse event” is an untoward medical occurrence that is associated with treatment with Compound, 1, or a pharmaceutically acceptable salt, hydrate, or solvate thereof. In one embodiment, an adverse event is selected from: leukopenia, constipation, diarrhea, nausea, abdominal pain, neutropenia, vomiting, back pain, and menstrual disorder. In one embodiment, an adverse event is heart block, for example, a first-degree atrioventricular heart block. In one embodiment, an adverse event is an acute heart rate reduction. In one embodiment, an adverse event is an abnormal pulmonary function test finding, such as an FEV1 below 80%, FVC. In one embodiment, an adverse event is macular edema.

IN NEED OF TREATMENT and IN NEED THEREOF: As used herein, “in need of treatment” and “in need thereof” when referring to treatment are used interchangeably to mean a judgment made by a caregiver (e.g. physician, nurse, nurse practitioner, etc.) that an individual requires or will benefit from treatment. This judgment is made based on a variety of factors that are in the realm of a caregiver's expertise, but that includes the knowledge that the individual is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

INDIVIDUAL: As used herein, “individual” means any human. In some embodiments, a human individual is referred to a “subject” or “patient.”

ACUTE HEART RATE REDUCTION: As used herein, “acute heart rate reduction” means a heart rate decrease from normal sinus rhythm of, for example, 10 or more beats per minute (bpm), such as less than about 5 bpm, e.g., less than about 4 bpm or less than about 3 bpm or less than 2 bpm, that is maximal within a few hours, for example 1-3 hours, after drug administration, and thereafter the heart rate returns towards the pre-dose value.

NORMAL SINUS RHYTHM: As used herein, “normal sinus rhythm” means the sinus rhythm of the individual when not undergoing treatment. The evaluation of normal sinus rhythm is within the ability of a physician. A normal sinus rhythm will generally give rise to a heart rate in the range from 60-100 bpm.

DOSE: As used herein, “dose” means a quantity of Compound 1, or a pharmaceutically acceptable salt, hydrate, or solvate thereof, given to the individual for treating or preventing the disease or disorder at one specific time.

THERAPEUTICALLY EFFECTIVE AMOUNT: As used herein, “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dose.

PHARMACEUTICAL COMPOSITION: As used herein, “pharmaceutical composition” means a composition comprising at least one active ingredient, such as Compound 1, including but not limited to, salts of Compound 1, whereby the composition is amenable to investigation for a specified, efficacious outcome. Those of ordinary skill in the art will understand and appreciate the techniques appropriate for determining whether an active ingredient has a desired efficacious outcome based upon the needs of the artisan.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed by Berge et al., Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

It is understood that when the phrase “pharmaceutically acceptable salts, solvates and hydrates” or the phrase “pharmaceutically acceptable salt, solvate, or hydrate” is used when referring to Compound 1, it embraces pharmaceutically acceptable solvates and/or hydrates of Compound 1, pharmaceutically acceptable salts of Compound 1, as well as pharmaceutically acceptable solvates and/or hydrates of pharmaceutically acceptable salts of Compound 1. It is also understood that when the phrase “pharmaceutically acceptable solvates and hydrates” or the phrase “pharmaceutically acceptable solvate or hydrate” is used when referring to Compound 1 that are salts, it embraces pharmaceutically acceptable solvates and/or hydrates of such salts.

It will be apparent to those skilled in the art that the dosage forms described herein may comprise, as the active component, either Compound 1, or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, DE), Avantium Technologies (Amsterdam) and Aptuit (Greenwich, CT).

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e., one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing and/or administering Compound 1, or a pharmaceutically acceptable salt, hydrate, or solvate thereof can be separated into two methods; one method reciting prescribing Compound 1, or a pharmaceutically acceptable salt, hydrate, or solvate thereof and the other method reciting administering Compound 1, or a pharmaceutically acceptable salt, hydrate, or solvate thereof. In addition, for example, a method that recites prescribing Compound 1, or a pharmaceutically acceptable salt, hydrate, or solvate thereof and a separate method of the invention reciting administering Compound 1, or a pharmaceutically acceptable salt, hydrate, or solvate thereof can be combined into a single method reciting prescribing and/or administering Compound 1, or a pharmaceutically acceptable salt, hydrate, or solvate thereof.

### Methods

Provided is a method of treatment of an S1P1 receptor-associated disorder in a subject, said method comprising administering (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, to said subject, wherein the subject is also being administered a hormone treatment.

In some embodiments, the administration does not affect the efficacy of the hormonal treatment.

In some embodiments, the administration results in a clinically non-relevant change in the C_{max,ss} and/or total (AUC_{tau,ss}) exposure measures of the hormonal treatment.

In some embodiments, the administration results in a clinically non-relevant change in one or more pharmacodynamic markers of ovarian activity. In some embodiments, the marker is chosen from follicle-stimulating hormone (FSH) level, luteinizing hormone (LH) level, estradiol level, progesterone level, sex hormone-binding globulin (SHBG) level, and transvaginal ultrasound (TVUS)/Hoogland score.

In some embodiments, the subject is female and the hormone treatment comprises a hormonal contraceptive.

In some embodiments, the hormonal contraceptive is monophasic.

In some embodiments, the monophasic hormonal contraceptive comprises a low dose (about 20 g) of an estrogen.

In some embodiments, the monophasic hormonal contraceptive comprises a regular dose (about 30 to about 35 g) of an estrogen.

In some embodiments, the monophasic hormonal contraceptive comprises a high dose (about 50 g) of an estrogen.

In some embodiments, the hormonal contraceptive is multiphasic.

In some embodiments, the hormonal contraceptive is a hormonal combined contraceptive product.

In some embodiments, the hormonal combined contraceptive product comprises one or more estrogens chosen from conjugated estrogens, mestranol, ethinyl estradiol and estradiol.

In some embodiments, the hormonal combined contraceptive product comprises one or more progestogen chosen from drospirenone, hydroxyprogesterone, megestrol, norethindrone, norethindrone acetate, ethynodiol diacetate, dydrogesterone, medroxy-progesterone acetate, norethynodrel, allylestrenol, lynoestrenol, medrogestone, norgestrienone, dimethiderome, ethisterone, cyproterone acetate, levonorgestrel, gestodene, desogestrel, etonogestrel, norgestimate, nestorone, dienogest, norelgestromin and drospirenone.

In some embodiments, the hormonal combined contraceptive product comprises ethinyl estradiol and a progestogen chosen from levonorgestrel, norgestrel, norelgestromin, drospirenone, norgestimate, desogestrel, etonogestrel, norethindrone acetate and norethindrone.

In some embodiments, the hormonal combined contraceptive product comprises ethinyl estradiol and levonorgestrel.

In some embodiments, the hormonal contraceptive product is progestogen-only contraceptive product.

In some embodiments, the hormonal contraceptive product comprises one or more progestogen chosen from drospirenone, hydroxyprogesterone, megestrol, norethindrone, norethindrone acetate, ethynodiol diacetate, dydrogesterone, medroxy-progesterone acetate, norethynodrel, allylestrenol, lynoestrenol, medrogestone, norgestrienone, dimethiderome, ethisterone, cyproterone acetate, levonorgestrel, gestodene, desogestrel, etonogestrel, norgestimate, nestorone, dienogest, norelgestromin and drospirenone.

In some embodiments, the hormonal contraceptive product comprises ethinyl estradiol and a progestogen chosen from levonorgestrel, norgestrel, norelgestromin, drospirenone, norgestimate, desogestrel, etonogestrel, norethindrone acetate and norethindrone.

In some embodiments, the hormonal contraceptive product is administered by a route chosen from oral, transdermal, intrauterine, subcutaneous, intramuscular, and intravaginal.

In some embodiments, the hormonal contraceptive product is an oral contraceptive composition, a subcutaneous implant, a contraceptive patch, or a vaginal ring.

In some embodiments, the hormone treatment comprises the administration of an estrogen.

In some embodiments, the subject is female and the estrogen is administered for the treatment of symptoms associated with menopause. In some embodiments, the symptoms associated with menopause are chosen from vasomotor symptoms, vulvar atrophy, and vaginal atrophy.

In some embodiments, the estrogen is administered for the treatment of hypoestrogenism due to hypogonadism, castration or primary ovarian failure.

In some embodiments, the estrogen is administered for the prevention of osteoporosis.

In some embodiments, the estrogen is administered for the treatment of breast cancer in subjects with metastatic disease.

In some embodiments, the estrogen is administered for the treatment of advanced androgen-dependent carcinoma of the prostate.

In some embodiments, the subject is male or non-binary and the hormone treatment comprises feminizing hormone therapy. In some embodiments, the feminizing hormone therapy comprises the administration of an estrogen and optionally, a progestogen.

In some embodiments, Compound 1, or a pharmaceutically acceptable salt, hydrate, or solvate thereof, is selected from: Compound 1, a calcium salt of Compound 1, and an L-arginine salt of Compound 1.

In some embodiments, Compound 1, or a pharmaceutically acceptable salt, hydrate, or solvate thereof, is an L-arginine salt of Compound 1.

In some embodiments, Compound 1, or a pharmaceutically acceptable salt, hydrate, or solvate thereof, is an anhydrous, non-solvated crystalline form of the L-arginine salt of Compound 1.

In some embodiments, Compound 1, or a pharmaceutically acceptable salt, hydrate, or solvate thereof, is a crystalline free-plate habit of the non-solvated L-arginine salt of Compound 1.

In some embodiments, Compound 1, or a pharmaceutically acceptable salt, hydrate, or solvate thereof, is an anhydrous, non-solvated crystalline form of Compound 1.

In some embodiments, Compound 1, or a pharmaceutically acceptable salt, hydrate, or solvate thereof, is administered orally.

In some embodiments, Compound 1, or a pharmaceutically acceptable salt, hydrate, or solvate thereof, is formulated as a capsule or tablet suitable for oral administration.

In some embodiments, Compound 1, or a pharmaceutically acceptable salt, hydrate, or solvate thereof, is administered once daily.

In some embodiments, the individual is administered an amount equivalent to about 0.5 to about 5.0 mg of Compound 1. In some embodiments, the individual is administered an amount equivalent to 2 mg of Compound 1. In some embodiments, the individual is administered an amount equivalent to 2.25 mg of Compound 1. In some embodiments, the individual is administered an amount equivalent to 2.5 mg of Compound 1. In some embodiments, the individual is administered an amount equivalent to 2.75 mg of Compound 1. In some embodiments, the individual is administered an amount equivalent to 3 mg of Compound 1. In some embodiments, the individual is administered an amount equivalent to 1 mg of Compound 1.

In some embodiments, the individual is administered Compound 1, or a pharmaceutically acceptable salt, hydrate, or solvate thereof at least one month, such as one month, two months, three months, four months, etc. In some embodiments, the individual is administered Compound 1, or a pharmaceutically acceptable salt, hydrate, or solvate thereof for least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the second time period is indefinite, e.g., chronic administration.

In some embodiments, the individual is administered an amount equivalent to 2 mg of Compound 1 for a first time period and subsequently an amount equivalent to 3 mg of Compound 1 for a second time period. In some embodiments, the first time period is at least one month, such as one month, two months, three months, four months, etc. In some embodiments, the first time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the second time period is at least one month, such as one month, two months, three months, four months, etc. In some embodiments, the second time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the second time period is indefinite, e.g., chronic administration. In some embodiments, the dosage for the first time period is not adjusted during the first time period. In some embodiments, the dosage for the second time period is not adjusted during the second time period.

In some embodiments, the individual is administered an amount equivalent to 3 mg of Compound 1 for a first time period and subsequently an amount equivalent to 2 mg of Compound 1 for a second time period. In some embodiments, the first time period is at least one month, such as one month, two months, three months, four months, etc. In some embodiments, the first time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the second time period is at least one month, such as one month, two months, three months, four months, etc. In some embodiments, the second time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the second time period is indefinite, e.g., chronic administration. In some embodiments, the dosage for the first time period is not adjusted during the first time period. In some embodiments, the dosage for the second time period is not adjusted during the second time period.

In some embodiments, the standard dose is administered without titration. In some embodiments, the standard dose is administered without titration; and the individual does not experience a severe related adverse event. In some embodiments, the standard dose is administered without requiring titration to avoid first-dose effect seen with other S1P receptor modulators.

In some embodiments, the dosage form is administered under fasted conditions. In some embodiments, the dosage form is administered under fed conditions.

In some embodiments, the method is non-gender specific.

In some embodiments, Compound 1, or a pharmaceutically acceptable salt, hydrate, or solvate thereof, is administered in combination with a second therapeutic agent or therapy, wherein the second therapeutic agent or therapy is selected from an IL-1beta inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a glucocorticoid, a proton pump inhibitor, a NSAID, allergen removal and diet management.

In some embodiments, Compound 1, or a pharmaceutically acceptable salt, hydrate, or solvate thereof, was previously administered at least one therapeutic agent or therapy, wherein the therapeutic agent or therapy is selected from an IL-1beta inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a glucocorticoid, a proton pump inhibitor, a NSAID, allergen removal and diet management.

In some embodiments, the individual is, or was, treated with an IL-1beta inhibitor. In some embodiments, the IL-1beta inhibitor is anakinra, rilonacept, or canakinumab.

In some embodiments, the individual is, or was, treated with an IL-5 inhibitor. In some embodiments, the IL-5 inhibitor is benralizumab, mepolizumab or reslizumab.

In some embodiments, the individual is treated with an IL-9 inhibitor.

In some embodiments, the individual is, or was, treated with an IL-13 inhibitor. In some embodiments, the IL-13 inhibitor is lebrikizumab, RPC4046, or tralokinumab.

In some embodiments, the individual is, or was, treated with an IL-17 inhibitor. In some embodiments, the IL-17 inhibitor is ixekizumab or brodalumab.

In some embodiments, the individual is, or was, treated with an IL-25 inhibitor.

In some embodiments, the individual is, or was, treated with a TNFα inhibitor. In some embodiments, the TNFα inhibitor is SIMPONI® (golimumab), REMICADE® (infliximab), HUMIRA® (adalimumab), or CIMZIA® (certolizumab pegol).

In some embodiments, the individual is, or was, treated with an eotaxin-3 inhibitor.

In some embodiments, the individual is, or was, treated with an IgE inhibitor. In some embodiments, the IgE inhibitor is omalizumab.

In some embodiments, the individual is, or was, treated with a prostaglandin D2 inhibitor.

In some embodiments, the individual is, or was, treated with an immunosuppressant. In some embodiments, the immunosuppressant is AZASAN® (azathioprine), IMURAN@(azathioprine), GENGRAF® (cyclosporine), NEORAL® (cyclosporine), or SANDIMMUNE@(cyclosporine). Immunosuppressants also may be referred to as immunosuppressives or immunosuppressive agents.

In some embodiments, the individual is, or was, treated with a proton pump inhibitor. In some embodiments, the proton pump inhibitor is omeprazole, pantoprazole, esomeprazole, or dexiansoprazole.

In some embodiments, the individual is, or was, treated with a glucocorticoid. In some embodiments, the glucocorticoid is UCERIS® (budesonide); DELTASONE® (prednisone), MEDROL® (methylprednisolone), or hydrocortisone. Glucocorticosteroids also may be referred to as glucocorticoid or corticosteroids.

In some embodiments, the individual is, or was, treated with a NSAID. In some embodiments, the NSAID is aspirin, celecoxib, diclofenac, diflunisal, etodolac, ibuprofen, indomethacin, ketoprofen, ketorolac, nabumetone, naproxen, oxaprozin, piroxicam, salsalate, sulindac, or tolmetin.

In some embodiments, the individual has had an inadequate response with, lost response to, or been intolerant to a conventional therapy. In some embodiments, the individual has had an inadequate response to conventional therapy. In some embodiments, the individual has lost response to conventional therapy. In some embodiments, the individual has been intolerant to conventional therapy. In some embodiments, the conventional therapy is selected from: an IL-1beta inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a glucocorticoid, a proton pump inhibitor, a NSAID, allergen removal and diet management. In some embodiments, the prior conventional therapy is referred to as prior treatment.

In some embodiments, the individual had an inadequate response with, lost response to, or was intolerant to the at least one therapeutic agent or therapy. In some embodiments, the individual has demonstrated an inadequate response to, loss of response to, or intolerance of to the at least one therapeutic agent or therapy. In some embodiments, the individual had demonstrated, over the previous 3-month period, an inadequate response to, loss of response to, or intolerance of the at least one therapeutic agent or therapy. In some embodiments, the individual had demonstrated, over the previous 6-month period, an inadequate response to, loss of response to, or intolerance of the at least one therapeutic agent or therapy. In some embodiments, the individual had demonstrated, over the previous 9-month period, an inadequate response to, loss of response to, or intolerance of the at least one therapeutic agent or therapy. In some embodiments, the individual had demonstrated, over the previous 1-year period, an inadequate response to, loss of response to, or intolerance of the at least one therapeutic agent or therapy. In some embodiments, the individual had demonstrated, over the previous 2-year period, an inadequate response to, loss of response to, or intolerance of the at least one therapeutic agent or therapy. In some embodiments, the individual had demonstrated, over the previous 3-year period, an inadequate response to, loss of response to, or intolerance of the at least one therapeutic agent or therapy. In some embodiments, the individual had demonstrated, over the previous 4-year period, an inadequate response to, loss of response to, or intolerance of the at least one therapeutic agent or therapy. In some embodiments, the individual had demonstrated, over the previous 5-year period, an inadequate response to, loss of response to, or intolerance of the at least one therapeutic agent or therapy.

In some embodiments, prior to the treatment, the individual will have been administered proton pump inhibitor therapy. In some embodiments, prior to the treatment, the individual had an inadequate response with, lost response to, or was intolerant to proton pump inhibitor therapy. In some embodiments, the individual will have been on a stable dose of proton pump inhibitor therapy for at least two months.

In some embodiments, prior to the treatment, the individual will not have severe strictures.

In some embodiments, the method further comprises monitoring for adverse events during the administration of Compound 1, or a pharmaceutically acceptable salt, hydrate, or solvate thereof, and optionally, interrupting or terminating the administration of Compound 1, or a pharmaceutically acceptable salt, hydrate, or solvate thereof.

In some embodiments, the treatment further comprises monitoring heart rate during the administration, monitoring pulmonary function during the administration, or monitoring liver function during the administration.

In some embodiments, the treatment further comprises monitoring heart rate during the administration.

In some embodiments, the treatment further comprises monitoring pulmonary function during the administration.

In some embodiments, the treatment further comprises monitoring liver function during the administration.

In some embodiments, the method reduces the incidence and severity of adverse events resulting from the treatment of a condition described herein.

In some embodiments, the adverse event is a serious adverse event.

In some embodiments, the serious adverse event is selected from leukopenia, constipation, diarrhea, nausea, abdominal pain, neutropenia, vomiting, back pain, and menstrual disorder.

In some embodiments, the method results in no serious adverse events.

In some embodiments, the standard dose is administered without substantially inducing an acute heart rate reduction or heart block in the individual.

In some embodiments, Compound 1, or a pharmaceutically acceptable salt, hydrate, or solvate thereof, is administered without causing a reduction of more than 6 bpm in heart rate.

In some embodiments, Compound 1, or a pharmaceutically acceptable salt, hydrate, or solvate thereof, is administered without a first-dose effect on heart rate as seen with other S1P receptor modulators. In some embodiments, Compound 1, or a pharmaceutically acceptable salt, hydrate, or solvate thereof, is administered without a first-dose effect on AV conduction as seen with other S1P receptor modulators.

### Example

A phase 1, open-label, repeat-dose, two-way, single-sequence study was conducted to evaluate the effect of Compound 1, or a pharmaceutically acceptable salt, hydrate, or solvate thereof, (“Study Drug”) on the pharmacokinetics and pharmacodynamics of a monophasic oral contraceptive (OC) in healthy premenopausal female subjects.

The primary objective of the study was to evaluate the pharmacokinetics (PK) of a monophasic OC (30 μg ethinylestradiol [EE] and 150 μg levonorgestrel [LVG]) alone and when co-administered with repeated doses of Study Drug in healthy premenopausal female subjects.

Secondary objectives of the study were:
To assess the concentrations of follicle stimulating hormone (FSH), luteinizing hormone (LH), estradiol, and progesterone during and after dosing with the monophasic OC alone and when co-administered with Study Drug in healthy premenopausal female subjects.
To evaluate transvaginal ultrasound (TVUS) and Hoogland scores on Days 1 and 11±1 in Period 1 and Period 2 in healthy premenopausal female subjects.
To evaluate sex hormone binding globulin (SHBG) concentrations on Days 1 and 21 in Period 1 and Period 2 in healthy premenopausal female subjects.
To evaluate the steady-state PK of Study Drug when co-administered with the monophasic OC in healthy premenopausal female subjects.
To assess complete blood count (CBC) including differential, absolute lymphocyte count lowering from baseline, and platelet count upon administration of monophasic OC alone and when co-administered with Study Drug in healthy premenopausal female subjects.
To evaluate the safety and tolerability of Study Drug when co-administered with the monophasic OC in healthy premenopausal female subjects.

The study population included healthy females of childbearing potential aged 18 to 40 years, inclusive, with a body mass index (BMI) of 18.0 to 32.0 kg/m², inclusive, at screening. Subjects had no history of irregular menses or pregnancy while on OCs. Subjects had no medical history of any arterial disease or thromboembolic conditions that predisposed to clotting disorders or other risk factors with OC use; and no second or third degree atrioventricular (AV) block, sick sinus syndrome without a functional pacemaker, periods of asystole for >3 seconds without an implanted cardiac defibrillator, or recurrent symptomatic bradycardia or recurrent cardiogenic syncope. Subjects had screening or check-in orthostatic vital signs with a pulse rate>50 bpm or systolic blood pressure (SBP)>90 mm Hg or diastolic blood pressure (DBP)>50 mm Hg; screening or check-in 12-lead PR interval≤220 ms; QTcF≤470 ms; and no history of congenital long QT syndrome. Subjects had no confirmed suppressed hematological function; aspartate aminotransferase (AST) and alanine aminotransferase (ALT)<2×upper limit of normal; total bilirubin<1× upper limit of normal; and estimated glomerular filtration rate>60 mL/min/1.73 m² at check-in.

Subjects were screened (approximately 28 days from signing consent to starting the OC pill pack) for eligibility. Enrolled subjects were synchronized (Day −90 to −1) in up to 3° C. cycles that could be extended or shortened (with a minimum of 14 days of OC use in a cycle) to achieve Day −8 as the last OC dose prior to the first day of OC dosing in Period 1 on Day 1. For enrolled subjects who had the onset of their menstrual cycle at a different timepoint, synchronization was achieved so that the onset of the menstrual cycles coincided at or around the same time in all of the enrolled subjects, thereby they maintained the same dosing schedule. This was accomplished through counselling the subjects and initiating the intake of OC pills based on each of their cycles and their expected scheduled dosing days.

A confirmatory predose PK blood draw was performed to check OC levels on Day 18±2 of every OC pill pack during the synchronization phase as applicable to specific subjects.

Twenty-four subjects were planned to be enrolled in Period 1 for a planned total of 21 evaluable subjects to complete the study.

### Period 1: OC PK and PD Evaluations

All subjects received monophasic OC once daily (qd) for 21 days (Days 1 to 21; reference treatment) followed by a 7-day OC dose-free period (Days 22 to 28).

On Day 1, baseline CBC, FSH, LH, estradiol, progesterone, TVUS, and SHBG were obtained, and then subjects received OC dosing. The TVUS were performed on Day −1 or Day 1. The OC PK was done on Days 21 to 22. Further PD assessments and safety evaluations were performed as described in the schedules of assessments and procedures.

Subjects were administered 2 mg Study Drug, specifically, the L-arginine salt of Compound 1, i.e., Compound 2, qd on Days 23 to 28. Subjects were fitted with a Holter monitor and 24-hour continuous electrocardiogram (ECG) recording was performed starting before the Study Drug dose on Day 23 of Period 1 until Day 24 of Period 1.

### Period 2: OC PK and PD and Study Drug PK Evaluations

All subjects were co-administered Study Drug 2 mg qd with OC for 21 days (Days 29 to 49; test treatment), followed by a 7-day dose-free period (Days 50 to 56). On Days 49 to 50, subjects underwent assessments for PK of Study Drug and OC. Further PD assessments and safety evaluations were performed as described in the schedules of assessments and procedures. Subjects were fitted with a Holter monitor and 24-hour continuous ECG recording was performed starting before the Study Drug dose on Day 49 of Period 2 until Day 50 of Period 2.

### Treatments Administered

Oral contraceptive was taken in the morning at approximately the same time for each dose day on the schedule of assessments.

Study Drug was taken alone at the same time every dose day for dosing on Days 23 to 28. Study Drug was taken with the OC (within 5 minutes) at the same time every dose day in Period 2 per the schedule of assessments.

Oral Study Drug 2 mg and OC 30 μg EE/150 μg LVG were administered with 240 mL noncarbonated water.

On Days 21 and 49, doses were administered following an overnight fast of at least 8 hours and followed by a standardized meal 4 hours postdose.

In Period 1, all subjects were assigned monophasic OC qd for 21 days (Days 1 to 21; reference treatment), followed by a 7-day OC dose-free period (Days 22 to 28). Subjects were administered 2 mg Study Drug qd on Days 23 to 28.

In Period 2, all subjects were assigned co-administration of Study Drug 2 mg qd with OC for 21 days (Days 29 to 49; test treatment), followed by a 7-day dose free period (Days 50 to 56).

### Pharmacokinetics:

Blood samples for plasma PK evaluation of EE, LVG, and Study Drug were collected. The following PK parameters were determined for EE, LVG, and/or Study Drug and, if warranted, metabolites thereof.
C_{max,ss} maximum plasma concentration at steady-state
C_{min,ss} plasma concentration at 24-hours postdose at steady-state
T_{max,ss} time to Cₘₐₓ at steady-state
AUC_{tau,ss} area under the plasma concentration-time curve (AUC) during the dosing interval (tau) at steady-state
CL_{SS}/F Apparent plasma clearance of drug after extravascular administration at steady-state

### Pharmacodynamics:

Evaluations for PD included FSH, LH, estradiol, progesterone, TVUS/Hoogland scores, SHBG, and CBC including differential, absolute lymphocyte count, and platelet count.

### Safety:

Subject safety and tolerability were monitored during the study using standard measures, including adverse event (AE) monitoring, orthostatic vital signs, 12-lead ECGs, continuous ECG recordings (Holter), clinical laboratory evaluations, physical examinations, and concomitant medication usage.

### Planned Endpoints:

### Primary PK Endpoints Included:

AUC_{tau,ss} and C_{max,ss} (EE, LVG)

### Primary PD Endpoints Included:

FSH, LH, estradiol, progesterone
TVUS, SHBG, Hoogland score
CBC including differential, absolute lymphocyte count, and platelet count Secondary PK endpoints w included ere:
AUC_{tau,ss} and C_{max,ss} for Study Drug and, if warranted, for M3 and M6
C_{min,ss}, T_{max,ss} for EE, LVG, Study Drug and, if warranted, for M3 and M6, and CL_{SS}/F for EE, LVG, and Study Drug only
Additional metabolites PK parameters such as parent to metabolite ratios for AUC_{tau,ss} and C_{max,ss} may also be calculated if warranted

### Safety and Tolerability:

Evaluations of AEs, orthostatic vital signs, 12-lead ECGs, continuous ECG recording (Holter), clinical laboratory evaluations, physical examinations, concomitant medications

### Results:

Only minor-to-moderate increases in EE and LVG peak (Cmax,ss) and total (AUCtau,ss) exposure measures were observed when the OC was coadministered with ETR, as compared to the OC alone (see Table). These changes were not considered clinically relevant. Similarly, PD markers of ovarian activity (FSH, LH, estradiol, progesterone, SHBG, and TVUS/Hoogland score) demonstrated no apparent clinically relevant impact of Study Drug on the PD performance of the OC.

TABLE
Geometric Least Square Mean Ratio (OC + ETR/OC Alone)
and 90% CI for PK Exposure Measures of the Pharmacologically
Active Components of the Evaluated Monophasic OC.

| | |
|---|---|
| C_{max, ss} | AUC_{tau, ss} |
| GMR (90% CI) | GMR (90% CI) |

| | |
|---|---|
| Component | [OC + ETR]/OC alone |

| | | |
|---|---|---|
| Ethinylestradiol (EE) | 111.95 | 124.05 |
| | (100.94-124.16) | (116.36-132.25) |
| Levonorgestrel (LVG) | 122.16 | 131.70 |
| | (115.01-129.76) | (126.85-136.73) |

AUC_{tau, ss}, area under the concentration-time curve during the dosing interval (tau) at steady state; CI, confidence interval; C_{max, ss}, maximum plasma drug concentration at steady state; GMR, geometric least squares mean ratio.

Study Drug steady-state plasma exposure measures (C_{max,ss}, C_{min,ss}, AUC_{tau,ss}) on Day 49 (Period 2; Study Drug+OC) appeared generally consistent with previous study findings for Study Drug when given alone and when also considering the female-only population enrolled in the present study.

Mean lymphocyte counts remained relatively stable in Period 1 (OC alone; reference treatment), but in Period 2 (Study Drug+OC; test treatment) were reduced by up to a mean of −56.7% (Day 49) from the mean baseline on Day 23, as expected due to the pharmacological effects of Study Drug. Mean lymphocyte counts on Day 56 returned to within 94% of the mean baseline (Day 23) counts, which was 7 days after discontinuation of study treatment.

From safety 12-lead ECG findings, the HR lowering effects of Study Drug were typically mild after the first dose (Day 23) and less impacted upon repeat dosing of Study Drug at steady-state (Day 49). No subject had an ECG with clinically significant abnormalities for 12-lead ECG parameter measurements including PR interval and QT interval on Days 23 or 49.

From continuous Holter ECG recording findings, the hHR lowering effects of Study Drug were typically mild after the first dose (Day 23) and less impacted upon repeat dosing of Study Drug at steady-state (Day 49).

All treatments were generally well tolerated. All treatment-emergent adverse events were mild or moderate. No abnormalities in laboratory parameters, vital signs, or 12-lead electrocardiogram assessments were noted.

### CONCLUSIONS

The PK and PD performance of the evaluated monophasic OC were not altered to a clinically relevant extent and thus contraceptive efficacy was maintained during coadministration with ETR.No safety or tolerability events of concern were detected during the study.

Those skilled in the art will recognize that various modifications, additions, substitutions, and variations to the illustrative examples set forth herein can be made without departing from the spirit of the invention and are, therefore, considered within the scope of the invention.

1. A method of treatment of an S1P1 receptor-associated disorder in a subject, said method comprising administering (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, to said subject, wherein the subject is also being administered a hormone treatment.

2. The method of claim 1, wherein the administration does not affect the efficacy of the hormonal treatment.

3. The method of claim 1, wherein the administration results in a clinically non-relevant change in the C_{max,ss} and/or total (AUC_{tau,ss}) exposure measures of the hormonal treatment.

4. The method of claim 1, wherein the administration results in a clinically non-relevant change in one or more pharmacodynamic markers of ovarian activity.

5. The method of claim 4, wherein the marker is chosen from follicle-stimulating hormone (FSH) level, luteinizing hormone (LH) level, estradiol level, progesterone level, sex hormone-binding globulin (SHBG) level, and transvaginal ultrasound (TVUS)/Hoogland score.

6. The method of claim 1, wherein the subject is female and the hormone treatment comprises a hormonal contraceptive.

7. The method of claim 6, wherein the hormonal contraceptive is monophasic.

8. The method of claim 7, wherein the monophasic hormonal contraceptive comprises a low dose (about 20 μg) of an estrogen.

9. The method of claim 7, wherein the monophasic hormonal contraceptive comprises a regular dose (about 30 to about 35 μg) of an estrogen.

10. The method of claim 7, wherein the monophasic hormonal contraceptive comprises a high dose (about 50 μg) of an estrogen.

11. The method of claim 6, wherein the hormonal contraceptive is multiphasic.

12. The method of claim 6, wherein the hormonal contraceptive is a hormonal combined contraceptive product.

13. The method of claim 12, wherein the hormonal combined contraceptive product comprises one or more estrogens chosen from conjugated estrogens, mestranol, ethinyl estradiol and estradiol.

14. The method of claim 12, wherein the hormonal combined contraceptive product comprises one or more progestogen chosen from drospirenone, hydroxyprogesterone, megestrol, norethindrone, norethindrone acetate, ethynodiol diacetate, dydrogesterone, medroxy-progesterone acetate, norethynodrel, allylestrenol, lynoestrenol, medrogestone, norgestrienone, dimethiderome, ethisterone, cyproterone acetate, levonorgestrel, gestodene, desogestrel, etonogestrel, norgestimate, nestorone, dienogest, norelgestromin and drospirenone.

15. The method of claim 14, wherein the hormonal combined contraceptive product comprises ethinyl estradiol and a progestogen chosen from levonorgestrel, norgestrel, noreigestromin, drospirenone, norgestimate, desogestrel, etonogestrel, norethindrone acetate and norethindrone.

16. The method of claim 14, wherein the hormonal combined contraceptive product comprises ethinyl estradiol and levonorgestrel.

17. The method of claim 6, wherein the hormonal contraceptive product is a progestogen-only contraceptive product.

18. The method of claim 17, wherein the hormonal contraceptive product comprises one or more progestogen chosen from drospirenone, hydroxyprogesterone, megestrol, norethindrone, norethindrone acetate, ethynodiol diacetate, dydrogesterone, medroxy-progesterone acetate, norethynodrel, allylestrenol, lynoestrenol, medrogestone, norgestrienone, dimethiderome, ethisterone, cyproterone acetate, levonorgestrel, gestodene, desogestrel, etonogestrel, norgestimate, nestorone, dienogest, norelgestromin and drospirenone.

19. The method of claim 12, wherein the hormonal contraceptive product comprises ethinyl estradiol and a progestogen chosen from levonorgestrel, norgestrel, norelgestromin, drospirenone, norgestimate, desogestrel, etonogestrel, norethindrone acetate and norethindrone.

20. The method of claim 6, wherein the hormonal contraceptive product is administered by a route chosen from oral, transdermal, intrauterine, subcutaneous, intramuscular, and intravaginal.

21-43. (canceled)
